(19) United States
(12) Patent Application Publication
(10) Pub. No.: US 2016/0310012 A1
(43) Pub. Date: Oct. 27, 2016

(54) CLOSED-LOOP VITAL SIGNS AND ENERGY HARVESTING SYSTEMS USING MICRO EVENTS FOR IMPROVED PERFORMANCE AND HYBRID WEARABLE/IMPLANTABLE APPLICATIONS

(71) Applicant: Peter MANKOWSKI, Waterloo (CA)

(72) Inventor: Peter MANKOWSKI, Waterloo (CA)

(21) Appl. No.: 15/136,825

(22) Filed: Apr. 22, 2016

Related U.S. Application Data
(60) Provisional application No. 62/176,917, filed on Apr. 25, 2015.

(51) Int. Cl. A61B 5/0205; A61B 5/07; A61B 5/00; A01K 29/00

(52) U.S. Cl. CPC A61B 5/0205; A01K 29/005; A61B 5/076; A61B 5/6831; A61B 5/7203; A61B 5/744; A61B 5/7275; A61B 5/7282; A61B 2503/40; A61B 2560/0214; A61B 2560/0257; A61B 5/0255

(57) ABSTRACT

An animal monitoring and energy harvesting system includes a wearable or implantable animal monitor sized and shaped to be worn by or implanted in an animal to be monitored. The animal monitor includes a sensor adapted to obtain a set of current animal physiology data associated with the animal, and a sensor processor coupled to the sensor. The sensor processor determines a current state of the animal based upon the set of current animal physiology data. The animal monitoring system also includes an animal monitor server in data communication with the animal monitor. The animal monitor server is configured to receive the current state of the animal. A computing device in data communication with the animal monitor server receives the current state of the animal from the animal monitor server and displays the current state of the animal being monitored.

### TECHNICAL FIELD

The embodiments herein relate to animal wearable or implantable systems.

### INTRODUCTION

Wearable devices for animals are becoming more prevalent in today's society. They provide access to various types of data that may be important for multitude of applications and those systems will continue adding new features. This poses challenges to provided sufficient power source that eliminates frequent re-charging.

### SUMMARY

According to some aspects, there is provided an animal monitoring and energy harvesting wearable system. The monitoring system includes a wearable or implantable animal monitor sized and shaped to be worn by or implanted in an animal to be monitored. The animal monitor includes at least one sensor adapted to obtain a set of current animal physiology data associated with the animal, and a sensor processor coupled to the sensor, the sensor processor being configured to determine a current state of the animal based upon the set of current animal physiology data. The animal monitoring system also includes: at least one animal monitor server in data communication with the animal monitor, the at least one animal monitor server being configured to receive the current state of the animal; and at least one computing device in data communication with the at least one animal monitor server, the at least one computing device configured to receive the current state of the animal from the at least one animal monitor server and display the current state of the animal being monitored.

In some aspects, the animal monitor is configured to: provide scanning capabilities to recognize following animal attributes: movement, heart rate, respiratory capacity.

In some aspects, the energy harvesting module is taking into the account the most predominant animal movement: walking, running, heart rate, chest cavity movement (breathing in/out) or other motion being generated by animal.

In some aspects, the system harvest energy from multiple planes: x, y, z. Three-dimensional (3D) spatial awareness of the energy harvesting is possible due use of multiple sensors and motion based capabilities.

In some aspects, the accelerometer, gyroscope and magnetonometer provide real time feedback to the energy harvester, controlling which plane presents the highest energy density for the piezo electric element. The displacement amount is directly proportional to the amount of movement and amount of energy collected. That 3D spatial awareness only enables energy harvester parts which will collect energy.

In some aspects, the energy harvester will only use one piezo element that collects energy from movement detected from sensors (accelerometer, gyroscope and magnetonometer). That decision to use one or multiple piezo elements requires system awareness of the directional force of the energy, intensity and inertia calculated per x, y and z planes.

In some aspects, the energy harvester will use multiple piezo elements that collect energy from movement detected from sensors (accelerometer, gyroscope and magnetonometer). That will occur if sensors detect movement which is not closely aligned with one 3D plane. In that case, two or more pieze electric elements are enabled, collecting energy.

In some aspects, the animal movement changes frequently and real time changes take place to only align the system to the movement to be used to extract the energy from animal motion.

In some aspects, the system will migrate from animal lung motion (respiratory) to heart rate (vital signs) to animal running/walking, constantly making decisions to maximize the amount of energy being harvested.

In some aspects, the animal motion has unknown origins and does not fit in any previous motion profiles. That movement might also be used and piezo elements will be aligned to it.

In some aspects, the animal motion is too unpredictable and sensors are unable to determine which plane to use for energy collection. Some complex movements are beyond what accelerometer, gyroscope and magnetonometer can model and in that state the system will continue enabling one piezo electric element only. During that scan state, microcontroller will read the amount of energy collected and writ it into its memory. After that, the system will move on to the next piezo electric energy element and repeat the process of reading the amount of energy. Having this ability to generate look up tables with energy registry per each element, 3D plane and minimum/maximum provided analytics for future decisions when similar event occurs.

In some aspects, the predominant movement from the animal is its heart rate and how each heart valve open/closes. In that case, the system will conduct a dual function of both using energy harvesting to collect energy and scanning for heart rate at the same time. In addition, the amount of piezo electric vibration/displacement is the information that is used by the main microcontroller to add accuracy, remove falls readings of the heart rate monitor unit.

In some aspects, the system will generate a look up table using both a heart rate monitor data and the piezo electric energy profiles. The purpose of blending both of them is greatly improved accuracy and filtering digital signal processor (DSP).

In some aspects, the system looks at the amount of energy harvested from the piezo electric element over each heart rate pulse. That information and the sampling rate is used as the filter removing noise and other unwanted artefacts from the raw heart rate monitor data logs.

The energy harvesting module adapts to the directional nature of movement to align itself to the angle that provides the highest motion and the largest energy collection module. That is accomplished by having multiple piezo electric elements that cover x, y and z planes.

In some aspects, at least one of the animal monitor server and the computing device is configured to: provide customization options to customize the animal movement profiles, generate analytics of motion over time and energy levels harvested per each look up table-case.

In some aspects, the at least one sensor includes: at least one heart rate monitor, energy harvester unit, accelerometer; gyroscope; and an altimeter.

In some aspects, the energy harvester includes at least three piezo electric elements covering 3D space as function of x, y and z planes.

In some aspects, the at least one energy harvesting power management unit is used to control one or multiple piezo electric elements.

In some aspects, at least one piezo electric power management controller is enabled at any point of time, which is decided by the main microcontroller and at least one sensor.

According to some other aspects, there is provided an animal monitor including: at least one sensor; a wireless transceiver; and at least one sensor processor coupled to the at least one sensor, the data storage device, and the wireless transceiver. The at least one sensor processor configured to obtain a set of current animal physiology data associated with the animal, determine a current state of the animal based upon the set of current animal physiology data, and transmit the current state of the animal using the wireless transceiver.

According to some aspects, the animal monitor further includes a data storage device having a library of animal states, each of the animal states being associated with at least one set of animal physiology data, wherein the at least one sensor processor is configured to determine the current animal state by selecting at least one of the animal states in the library of animal states based upon the current set of animal data.

According to some other aspects, there is provided a computer implemented method for monitoring an animal, the method including: obtaining a set of current animal physiology data associated with the animal; determining a current state of the animal based upon the set of current animal physiology data; transmitting the current state of the animal using the wireless transceiver to at least one animal monitor server; receiving the current state of the animal from the at least one animal monitor server at a computing device; and displaying the current state of the animal being monitored at the computing device.

### BRIEF DESCRIPTION OF THE DRAWINGS

Various embodiments will now be described, by way of example only, with reference to the following drawings, in which:

FIG. 1 is a schematic diagram illustrating components of an animal wearable unit;

FIG. 2 is a schematic diagram illustrating exemplary types of information that are processed and generated by the processor shown in FIG. 1;

FIG. 3 is a schematic diagram illustrating exemplary modules that may be provided by the system shown in FIG. 2 for the purpose of energy harvesting enhancements;

FIG. 4 is a schematic diagram illustrating a number of exemplary factors that may be involved in energy harvesting decisions in module shown in FIG. 3;

FIG. 5 is a schematic diagram illustrating steps of a computer-implemented method for monitoring animal's heart rate and enhancements based on piezo elements energy production according to some embodiments.

### DETAILED DESCRIPTION

This disclosure describes a combination of sensors and energy harvesting techniques as a closed-loop module that, when combined, adds an array of new capabilities and increased accuracy levels to animal monitoring.

This disclosure blends three techniques together: motion based models, heart rate monitor and 3D energy harvesting as one closed-loop application.

Animals, such as pets, large animals or livestock, can be very important to their owners. Owners are concerned with wellbeing of their animals and may be interested in knowing how their pets are doing at all times. However, it is often impractical for owners to monitor their animals or/and livestock around the clock. In that case, the new techniques discussed herein provide the ability to recognize animal movement, calibrate to each motion component and harvest energy as a continuous current collection to enhance battery life and accuracy of vital signs scanning.

Animal motion includes a multitude of kinetic movements and micro-events hidden from outside world. Techniques discussed herein extract energy by harvesting motion-based energy from not single but multiple sources available at any point of time.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments generally described herein.

Furthermore, this description is not to be considered as limiting the scope of the embodiments described herein in any way, but rather as merely describing the implementation of various embodiments as described.

In some cases, the embodiments of the systems and methods described herein may be implemented in hardware or software, or a combination of both. In some cases, embodiments may be implemented in one or more computer programs executing on one or more programmable computing devices comprising at least one processor, a data storage device (including in some cases volatile and non-volatile memory and/or data storage elements), at least one input device, and at least one output device.

In some embodiments, each program may be implemented in a high level procedural or object oriented programming and/or scripting language to communicate with a computer system. However, the programs can be implemented in assembly or machine language, if desired. In any case, the language may be a compiled or interpreted language.

In some embodiments, the systems and methods as described herein may also be implemented as a non-transitory computer-readable storage medium configured with a computer program, wherein the storage medium so configured causes a computer to operate in a specific and predefined manner to perform at least some of the functions as described herein.

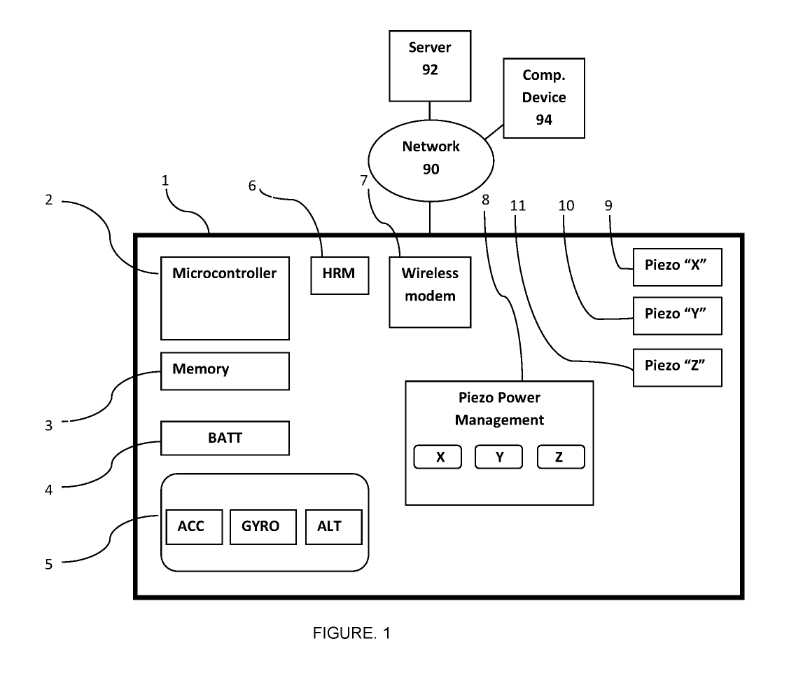

Referring now to FIG. 1, illustrated therein is an animal monitoring system 1 according to some embodiments. The system 1 may be used for monitoring various types of animals, including household pets (e.g. dogs and cats), horses, exotic zoo animals and livestock. The system 1 includes a wearable animal monitor 1 in wireless communication with a network 90 such as one or more of a cellular network, wifi, BTL, or other wireless standards for communications with an animal monitoring server 92. Microcontroller 2 provides a complete system management, memory 3 read/writes, battery 4 is shared with all functional blocks of the system.

The animal monitoring system 1 communicates monitored animal data to the animal monitoring server 92, which communicates the data to a computing device 94 connected to the network 90. The computing device 94 can be a laptop/desktop computer, smartphone, tablet computer, or similar configured for display or other outputting of the data. Various computing devices 94 operated by various owners or caregivers of animals bearing various animal monitoring systems 1 can be provided.

Set of motion sensors 5 accelerometer, gyroscope and altimeter provide movement based awareness in 3D space.

Piezo Power Management 8 controls all three piezo electric energy harvesting elements; 9-“X”, 10-“Y” and 11-“Z”.

The animal monitor 1 is sized and shaped to be worn by an animal under test and in some cases is installed on neck collar, animal harness specific to the breed of animal, or any mounting piece of generally used method of managing/controlling the animal. Eg. Horses head harness, saddle, or others. In other embodiments, the system 1 is configured to be implanted in the body of the animal.

The animal monitor 1 may be worn at a single location on the animal such as the animal's neck or multiple units can be installed and used simultaneously installed at multiple animal body locations. Animal wearable can be used independently of other units also present during the test.

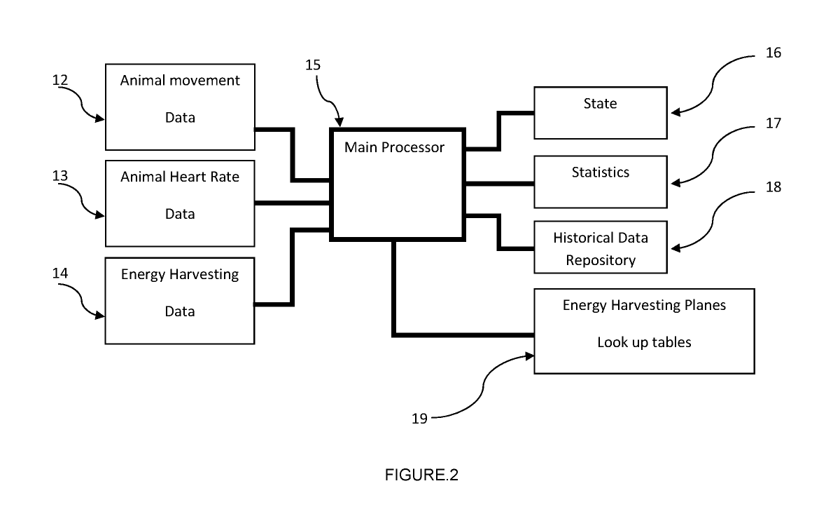

Referring now to FIG. 2, illustrated therein are exemplary components of the animal monitor 1 according to some embodiments. Power for the operation of the animal monitor 1 may be provided from one or more suitable power sources. For example, a rechargeable Lithium-Ion battery and suitable hardware configuration for recharging the battery (e.g. a printed circuit board with recharging functionality and recharging hardware such as a charging dock, wireless charging) may be provided.

The device 1 may also be configured to withstand adverse conditions such as wetness. For example, the monitor 1 may be water resistant or waterproof.

The animal wearable in FIG. 2 scans information from animal movement 12, animal heart rate 13 and energy harvesting data 14.

In some embodiments, there may be more than one valid inputs to the main processor 15

It should be understood that inputs 12, 13 and 14 are main source of raw information which is used to provide multiple alarms, notifications, timers, routines and other means of communication with other parts of the system.

The main processor continuously builds system process states 16, event statistics 17, historical data repository 18 and energy harvesting planes look up tables 19.

The sensor processor 15 is also operatively coupled to a wireless modem 7. The wireless modem 7 enables wireless transmission of the animal physiology data and or other information from the animal monitor 1. The wireless modem 7 may include a WIFI transceiver, a Bluetooth™ transceiver, Bluetooth™ low energy (BLE) transceiver or any other suitable wireless transceiver.

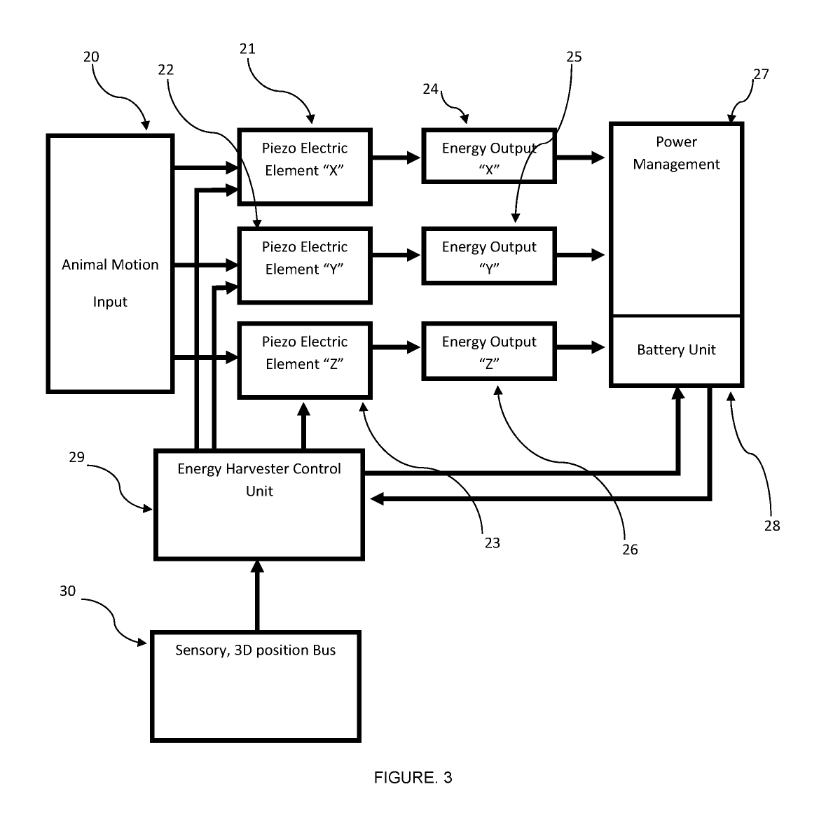

Referring now to FIG. 3, illustrated therein are various types of information that are processed and generated by the sensor processor 15 according some embodiments. The animal motion input 20 could be but is not limited to walking, running, jumping, respiratory chest cavity movement, heart movement and other movements produced by animals.

To determine the optimal method of harvesting kinetic energy, all three piezo electric element and initially enables and connected to the source of movement. Piezo electric element 21, 22 and 23 deliver various current outputs based on the relative unit displacement amount and individually feed the power management unit 27 through output X-24, Y-25 and Z-26.

In many cases, it will not be possible to obtain an exact and optimized control mechanism to decide which piezo element is the best under changing conditions. The power management 27 makes those decisions in real time, feeding on board battery 28 with energy load from one or multiple piezo elements, based on but not limited to sensory feed responses.

To increase the usability of the animal wearable unit 1, the energy harvesting control unit 29 controls which piezo element is enabled at any point of time during the operation of device 1.

In some embodiments, the energy harvesting control unit 29 collaborates with the power management unit 27 to maximize the amount of energy from animal motion.

The training and the initial calibration of the system provides multiple means of decision making to decide from which direction the maximum movement will enhance the animal wearable 1 to maximize energy harvesting.

Additionally, having three motion based sensors 5 allows the animal monitor to be aware of the x, y and z axis and permits re-calibration of the system in real time to account for variations in the sensor position. This recalibration can occur periodically in the background, can be enabled based on interrupt, timer, can be based on a changing motion profile, or similar.

In many cases, the sensory, 3D position Bus is aware of which 3D plane presents the best opportunity to harvest maximized amount of power. Accelerometer, gyroscope and magnetonometer 5 allow for fast and dynamic changes of piezo electric set up based on animal movement complexity.

In addition to an ongoing calibration and x-y-z sensor based positioning calculations, occasional scan of other configurations are being implemented but those are not visible to a user and being part of the embedded software part.

Now, that the link between 3D space and piezo element has been established, the system is described for its heart rate scanning capabilities.

In some modes of operation, energy harvesting elements are actually performing a dual function of energy collection and scan of heart rate.

The displacement-bending profile for one or multiple piezo elements is used to recognize and calculate animal heart rate profiles.

That is accomplished by understanding the directional nature of heart movement, energy density, and other heart produced motions.

In summary of FIG. 3, an ability to develop a closed loop system when sensors 5 collaborate with three piezo electric elements allows for better energy extraction and dynamic thermal adjustments of the system 1. In addition, as the system harvest energy, it also recognizes and tunes to animal heart rate to improve to overall vital signs accuracy.

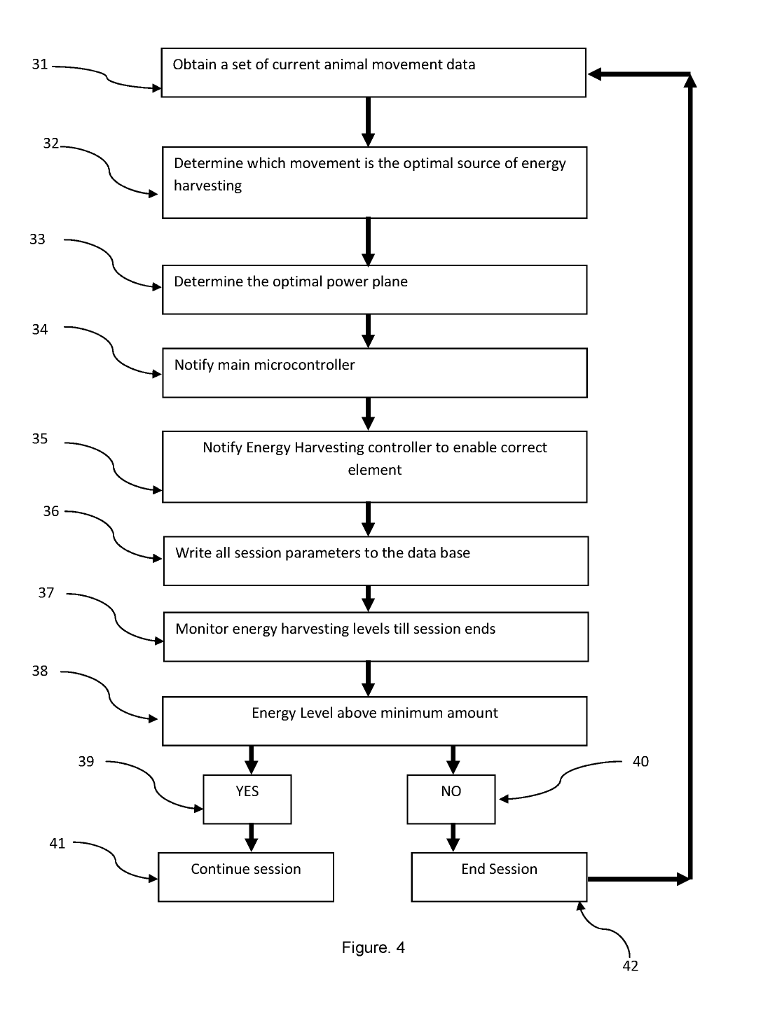

Referring now to FIG. 4, illustrated are a number of exemplary factors that may be activated during energy harvesting session, calibration and a back end activities with relations to animal movement such as walking/running, heart rate and respiratory chest movement.

At the beginning of the session, system obtains a set of current animal movement data 31. That information determines which movement 32 is the optimal source of energy harvesting. System looks at energy density, amount of displacement, frequency and power planes coordinates. The system recognizes but is not limited to animal walking, running and other motion related activities. In addition, animal heart rate and heart movement per pulses and animal lung movement during breathing are also used.

While animal movement is dynamic and has elements of unknown, the animal wearable system 1 determines the optimal device power plane 33 and is aware of device 1 3D coordinates as x-y-z values.

The main microprocessor 15 is notified via event 34, as well as energy harvesting controller 29 by software event 35.

All session parameters 36 are stored in the log session and system monitors energy levels being transferred to the battery as event 37.

The mechanism that provides a decision if the current session is to be continues is 38, with “YES 39 and “NO” 40 forks leading to one of two possible outcomes. 41 sessions meets all parameters and is to be continued, or “End session” 42 which forces repeating process an event 31 obtain a set of current animal movement parameters by initializing the process.

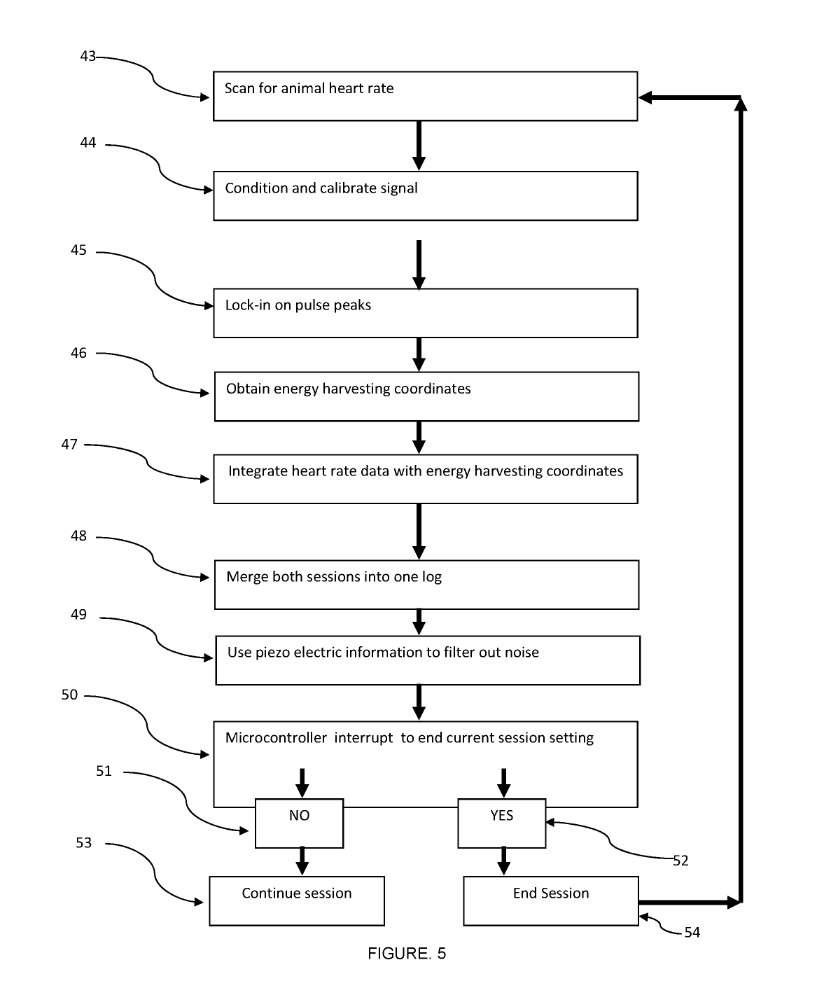

Referring now to FIG. 5, illustrated is a number of exemplary factors that may be activated during animal heart rate scan, event 43.

Initial signal conditioning, event 44 is activated and a preliminary search for pulse begins.

After a pulse pattern is found and qualified over several cycles, system locks-in pulse peaks using event 45.

At that time an animal heart rate has been acknowledged but an additional method is being called, 47 energy harvesting coordinates. As the energy element produces energy from mechanical stress, the amount of energy produced per each event is used to enhance heart rate results by merging both by software event 48

Most important enhancement from the piezo electric profile is noise cancellation. Event 49.

The session can be interrupted or reset by software event 50. Two possible outcomes; “NO” 51 and “YES” 52 are in place.

If event 51 NO, session continuous uninterrupted.

If event 52 YES, the process migrates to the software event 43; initial scan for animal heart rate.

The present invention applies to monitoring of animals, such as pets, horses, large animals or livestock, and even humans (adults or children). Owners/parents/caregivers may benefit from the invention by being able to better monitor the wellbeing of the monitored individual.

While the foregoing provides certain non-limiting example embodiments, it should be understood that combinations, subsets, and variations of the foregoing are contemplated. The monopoly sought is defined by the claims.

I claim:

1. An animal monitoring system, the system comprising:
a animal monitor sized and shaped to be worn by or implanted in an animal to be monitored, the animal monitor including:
at least one sensor adapted to obtain a set of current animal physiology data associated with the animal,
at least one energy harvesting component aligned with animal movement power planes,
heart rate monitor function that collaborates with both energy harvesting functions as well as accelerometer, gyroscope and altimeter sensors, and
a sensor processor coupled to the sensor, the sensor processor being configured to determine a current state of the animal based upon the set of current animal physiology data; and
at least one animal monitor server in data communication with the animal monitor, the at least one animal monitor server being configured to receive the current state of the animal;
at least one computing device in data communication with the at least one animal monitor server, the at least one computing device configured to receive the current state of the animal from the at least one animal monitor server and display the current state of the animal being monitored.

2. The system of claim 1, wherein the animal monitor is configured to:
provide a library of animal states, each of the animal states being associated with at least one set of animal physiology data; and
determine the current animal state by scanning motion, energy harvesting profiles and other animal functions as one closed-loop system.

3. The system of claim 2, wherein the heart rate monitor can be configured to operate in a stand-alone mode, or in synchronization mode with the energy harvesting function for added accuracy, filtering noise and other vital signs performance improvements.

4. The system of claim 2, wherein the motion of animal is used to generate awareness of x-y-z directional power planes and calculations which single plane, or multiples will provide the largest amount of energy.

5. The system of claim 2, wherein the animal monitor is configured to use the energy harvesting components X, Y, Z for a dual functionality; energy collection and a scan of animal heart rate functions.

6. The system of claim 5, wherein the log of the animal movement is stored in the memory, local or remote servers for the purpose of energy harvesting calibration.

7. The system of claim 5, wherein the at least one piezo electric energy harvesting element has been activated by animal motion and is displacement, amount of stress and frequency at which it reacts to the animal movement is used for scanning multiple vital signs.

8. The system of claim 5, wherein the animal monitor's constructs a long term analytics look up tables with animal movement and matching energy harvesting profiles.

9. The system of claim 5, wherein a multiple animal movements are being monitored simultaneously for the purpose of determining amount of kinetic energy amount per movement.

10. The system of claim 5, wherein a multiple animal movements are being monitored simultaneously for the purpose of determining which profile is useful for animal heart rate monitor performance improvements.

11. The system of claim 10, wherein a system avoids measuring animal vital signs during particular animal movement or multiple motions, more complex set of movements knowing the probability of accurate data will be low.

12. The system of claim 5, wherein energy harvesting amount per x, y and z planes is used in addition to the main heart rate monitor for digital signal processing improvements during raw data computation, noise removal and other DSP related functionality.

13. The system of claim 1, wherein the animal monitor operable in a home mode and an away mode, the animal monitor being in wireless data communication with the hub when operating in the home mode and the animal monitor being in wireless data communication with the computing device when operating in the away mode.

14. The system of claim 1, wherein the at least one computing device is configured to display a current state of the animal by animating an avatar being used to represent the animal being monitored.

15. The system of claim 5, wherein when a current state of the animal is not generated from the current set of animal physiology data, a predicted state of the animal is determined by reconstructing relevant information by fragments of information collected from all sources available: movement, energy harvesting profiles in x, y and z planes.

16. The system of claim 1, wherein at least one of the animal monitor server and the computing device is configured to:
provide ability to store animal movement profiles synchronized with sensory data responses to recall those at later time for faster decision making;
select one or multiple energy harvesting piezo electric modules by retrieving a past information from systems memory by similarities;
provide the generic heart rate monitor as a stand-alone feature;
provide the energy harvesting based heart rate monitor feature as a stand-alone feature; and
provide a hybrid heart rate monitor animal feature by combining energy harvesting and the standard heart tare monitor functions.

17. The system of claim 1, wherein the at least one sensor further includes a heart rate sensor with or without active assistance from the energy harvesting function.

18. The system of claim 1, wherein the animal monitor is a device having attachment mechanisms for attaching the device to an animal collar, harness or any other accessories for this particular animal, livestock, wildlife or others.

19. An animal monitor comprising:
at least one sensor;
a wireless transceiver; and
at least one sensor processor coupled to the at least one sensor, the data storage device, and the wireless transceiver, the at least one sensor processor configured to:
obtain a set of current animal physiology data associated with the animal,
determine a current state of the animal based upon the set of current animal physiology data, and
transmit the current state of the animal using the wireless transceiver.

20. The animal monitor of claim 14, further comprising a data storage device having a library of animal states, each of the animal states being associated with at least one set of animal physiology data, wherein the at least one sensor processor is configured to determine the current animal state by selecting at least one of the animal states in the library of animal states based upon the current set of animal data.

21. A computer implemented method for monitoring an animal, the method comprising:
obtaining a set of current animal physiology data associated with the animal using an animal monitor;
determining a current state of the animal based upon the set of current animal physiology data;
transmitting the current state of the animal using the wireless transceiver to at least one animal monitor server;
receiving the current state of the animal from the at least one animal monitor server at a computing device; and
displaying the current state of the animal being monitored at the computing device.
